(19) United States
(12) Patent Application Publication
(10) Pub. No.: US 2012/0283280 A1
(43) Pub. Date: Nov. 8, 2012

(54) IMIDAZOTETRAZINONE-BASED COMBI-MOLECULES

(76) Inventor: Bertrand Jean-Claude, Montreal (CA)

(21) Appl. No.: 13/450,056

(22) Filed: Apr. 18, 2012

Related U.S. Application Data
(60) Provisional application No. 61/476,478, filed on Apr. 18, 2011.

(51) Int. Cl. C07D 487/04; A61P 35/00; A61K 31/517

(52) U.S. Cl. 514/266.23; 544/179

(57) ABSTRACT

A series of new chemical agents that demonstrate anti-tumor activity are described herein. The new chemical agents exhibit a dual mode of anti-tumor action: blocking epidermal growth factor receptor (EGFR) mediated signal transduction and damaging DNA by alkylation.

### CROSS REFERENCE TO RELATED APPLICATIONS

This application claims the benefit of U.S. provisional application Ser. No. U.S. 61/476,478, filed on Apr. 18, 2011

### FIELD

The present disclosure broadly relates to novel chemical agents demonstrating anti-tumor activity. More specifically, but not exclusively, the present disclosure relates to molecules referred to as “combi-molecules” that exhibit a dual mode of anti-tumor action; blocking epidermal growth factor receptor (EGFR) mediated signal transduction and damaging DNA by alkylation. The present disclosure also relates to imidazotetrazinone-based combi-molecules and to a process for producing same.

### BACKGROUND

Cancer is a disease state characterized by the uncontrolled proliferation of genetically altered tissue cells. There have been several chemotherapeutic approaches developed to target cancer. These include anti-mitotic and alkylating agents, anti-metabolites and anti-tumor antibiotics. Such anti-cancer agents act preferentially on rapidly proliferating cells such as cancer cells.

The utility of most of the anti-tumor agents currently in clinical use is limited by their lack of selectivity for tumour cells. Moreover, these agents often exhibit a low therapeutic index, which further limits their efficacy in the clinic. Furthermore, in view of their low therapeutic index, administration of dosages that encroaches the patient tolerable limit is often required for achieving any significant therapeutic effect.

Acquired resistance mediated by DNA repair enzymes has often imposed severe limitations on the use of DNA-interactive agents and, in many cases, useful clinical anti-tumor activity could not be observed with the administration of multiple anti-cancer drugs having different mechanisms of action.

Prostate cancer (PCa) is a major cause of death in North America [1]. Therapeutic options for metastatic prostate cancer, however, are scant [2]. Advanced prostate cancer is initially treated by androgen ablation. Although effective, androgen ablation controls metastatic prostate cancer for approximately 18 months. Thereafter, the disease progresses to a stage where it is referred to as hormone-refractory prostate cancer (HRPC) [3]. Docetaxel-based chemotherapy provides a modest survival benefit in patients with metastatic HRPC. Almost all patients ultimately progress to an advanced stage of the disease and can experience severe toxic side effects upon treatment with docetaxel [4, 5].

The overexpression and dysfunction of tyrosine kinases (TKs) has been extensively studied and is now considered the major functional difference between normal and tumor cells. Because of their significant involvement in tumor progression, overexpressed receptor TKs have now become the targets for drug design and selective chemotherapeutic interventions. EGFR targeting therapy has now pervaded anti-cancer drug development. In vitro and in vivo studies have shown that inhibition of EGFR mediated signal transduction resulted in growth inhibition and reduced tumor invasiveness and metastasis [6].

However, despite the significant activity of EGFR TK inhibitors in preclinical models, Phase II clinical trials only confirmed their well-established tolerability profile. No single-agent activity was observed with these agents in HRPC [7, 8].

Further preclinical studies have confirmed that combining an EGFR inhibitor with a cytotoxic agent (e.g. paclitaxel) can induce synergistic therapeutic effects against androgen-independent prostate cancer cells [9]. However, the latter agent lacks tumor selectivity and is the cause of severe toxicity. The toxicity associated with drug combination therapies constitutes a major deterrent in their Further use as acceptable clinical therapies for the treatment of prostate cancer.

Mitozolomide (MTZ), the first lead drug of the imidazotetrazinone class of alkylating agents failed in phase II clinical trials due to acute, unpredictable and unacceptable myelotoxicity, particularly thrombocytopenia [10, 11]. The cytotoxic mechanism of this class of agents is based on the generation of interstrand DNA cross-links produced by the reaction of the cyclic intermediate N-1-O6-ethanoguanine with the cytosine residue of the opposite DNA strand. As this blocks DNA replication, it is likely to be cytotoxic for the cells [12]. Moreover, the potency and clinical activity of drugs similar to mitozolomide (e.g. temozolomide and dacarbazine) is severely hampered by the resistance mechanism of prostate cancer cells that express O⁶-alkylguanine transferase (AGT), a DNA repair enzyme that removes the O⁶-alkyl group by transferring it onto its own cysteine residue [13].

Autocrine expression of growth factors including EGF and TGFa, and the upregulation of EGFR and other members of the erbB family often contributes to the aggressiveness of prostate tumors and their reduced sensitivity to chemotherapeutic drugs, a phenomenon attributed to the ability of the EGFR to activate anti-apoptotic signaling.

Combi-molecules capable of blocking EGFR-mediated signaling and inducing cytotoxic DNA damage have been previously reported by Jean-Claude et al. [14, 15]. It was surmised that the action of an EGFR TK inhibitor could be enhanced by combining it with the action of a cytotoxic drug within the framework of a single molecule termed “combi-molecule”.

The present disclosure refers to a number of documents, the content of which is herein incorporated by reference in their entirety.

### SUMMARY

The present disclosure relates to combi-molecules exhibiting epidermal growth factor receptor (EGFR) inhibitory properties and DNA alkylating properties.

In an embodiment, the present disclosure relates to a molecule of Formula I or a pharmaceutically acceptable salt or a prodrug thereof:

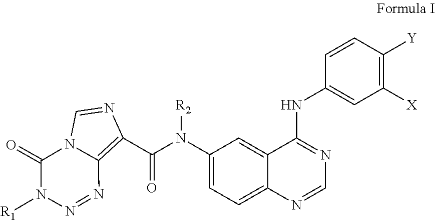

wherein:

X is selected from the group consisting of H, F, Cl, Br, I Me and C≡CH;

Y is selected from the group consisting of H, F, Br and Cl;

R₁ is selected from the group consisting of H, alkyl; ClCH₂CH₂—, HOCH₂CH₂—, MsOCH₂CH₂—, TsOCH₂CH₂—;

R₂ is selected from the group consisting of H, alkyl, ClCH₂CH₂—, HOCH₂CH₂—, MsOCH₂CH₂—, TsOCH₂CH₂—,

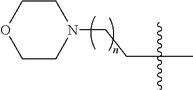

and

n is an integer ranging from 1 to 6.

In a further embodiment, the present disclosure relates to a molecule of Formula Ia (JDF12) or a pharmaceutically acceptable salt or a prodrug thereof:

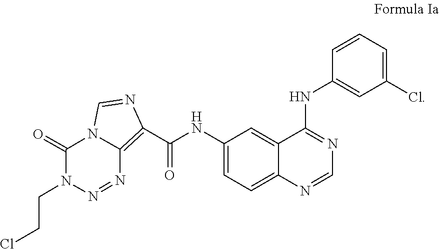

In an embodiment, the present disclosure relates to a molecule of Formula II or a pharmaceutically acceptable salt or a prodrug thereof:

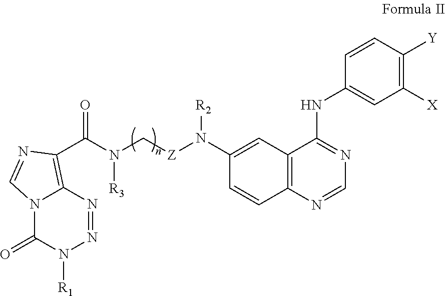

wherein:

X is selected from the group consisting of H, F, Cl, Br, I, Me and C≡CH;

Y is selected from the group consisting of H, F, Br and Cl;

Z is selected from the group consisting of —CO— and —CH₂—;

R₁ is selected from the group consisting of H, alkyl; ClCH₂CH₂—, HOCH₂CH₂—, MsOCH₂CH₂—, TsOCH₂CH₂—;

R₂ and R₃ are independently selected from the group consisting of H, alkyl, ClCH₂CH₂—, HOCH₂CH₂—, MsOCH₂CH₂—, TsOCH₂CH₂—, and

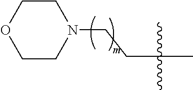

n and m are integers ranging from 1 to 6.

In an embodiment, the present disclosure relates to a molecule of Formula IIa or a pharmaceutically acceptable salt or prodrug thereof:

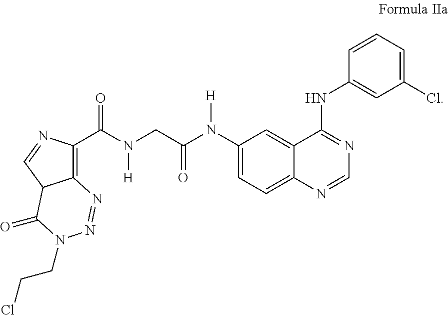

In an embodiment, the present disclosure relates to a molecule of Formula IIa or a pharmaceutically acceptable salt or prodrug thereof:

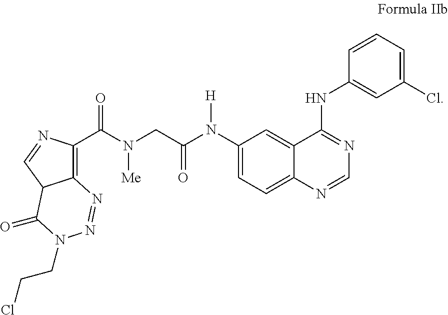

In an embodiment, the present disclosure relates to a method of treating a patient afflicted with a condition characterized by uncontrolled cell proliferation, the method comprising administering to the patient a therapeutically effective amount of one or more of the combi-molecules as disclosed herein.

In an embodiment, the present disclosure relates to a method of reducing proliferation of/or inducing cell death in neoplastic cells, the method comprising contacting the neoplastic cells with one or more of the combi-molecules as disclosed herein.

In an embodiment, the present disclosure relates to the use of one or more of the combi-molecules as disclosed herein in the manufacture of a medicament for treating a condition characterized by uncontrolled cell proliferation.

In an embodiment, the present disclosure relates to a pharmaceutical composition comprising an effective amount of one or more of the combi-molecules as disclosed herein in association with one or more pharmaceutically acceptable carriers, excipients or diluents.

In an embodiment, the present disclosure relates to an admixture comprising an effective amount of one or more of the combi-molecules as disclosed herein in association with one or more pharmaceutically acceptable carriers, excipients or diluents.

The foregoing and other objects, advantages and features of the present disclosure will become more apparent upon reading of the following non restrictive description of illustrative embodiments thereof, given by way of example only with reference to the accompanying drawings.

### BRIEF DESCRIPTION OF THE DRAWINGS

In the appended drawings:

FIG. 1 shows the percent inhibition of EGFR by JDF12, JDF04R and Mitozolomide. Poly(L-glutamic acid-L-tyrosine, 4:1) (PGT) substrate phosphorylation was detected using an anti-phosphotyrosine antibody. Each point represents at least 2 experiments run in triplicate.

FIG. 2 shows the inhibition of EGF-stimulated EGFR autophosphorylation by JDF-12, JDF04R and Mitozolomide in the DU145 cell line. DU145 cells were starved for 24 h and preincubated with the indicated concentrations of the drugs over a period of 2 hours followed by the addition of 50 ng/ml EGF over a period of 15 min. Western blotting was performed with an anti-phosphotyrosine antibody (1:1000 dilution). Membranes were stripped of anti-phosphotyrosine and reprobed for EGFR with anti-EGFR antibodies.

FIG. 3a shows a picture of DU145 cells exposed to various concentrations of JDF12, comets were visualized and examined using fluorescence microscopy at 330× magnification; FIG. 3b shows the quantization of DNA damage using the alkaline comet assay; the tail moment was used as a parameter for the detection of DNA damage in DU145 cells exposed to MTZ, JDF12 and JDF04R.

FIG. 4a-d shows the antiproliferative effects of JDF-12, JDF04R, MTZ and Iressa in DU145 cells (FIG. 4a), LNCaP-WT cells (FIG. 4b), LNCaP-EGFR cells (FIG. 4c) and LNCaP-erbB2 cells (FIG. 4d). Cells were treated with JDF-12, JDF04R, MTZ and Iressa over a period of 6 days (continuous exposure). Growth inhibition was measured using the SRB assay. Each point represents at least two independent experiments run in triplicate.

FIG. 5a-d shows the antiproliferative effects of JDF-12, MTZ/JDF04R (equimolar combination) and MTZ/Iressa (equimolar combination) in DU145 cells (FIG. 5a), LNCaP-WT cells (FIG. 5b), LNCaP-EGFR cells (FIG. 5c) and LNCaP-erbB2 cells (FIG. 5d). Cells were treated with JDF-12, MTZ+JDF04R (1:1) or MTZ+Iressa (1:1) over a period of 6 days (continuous exposure). Growth inhibition was measured using the SRB assay. Each point represents at least two independent experiments run in triplicate.

FIG. 6a-d shows the antiproliferative effects of JDF-12, MTZ/JDF04R (equieffective combination) and MTZ/Iressa (equieffective combination) in DU145 cells (FIG. 6a), LNCaP-WT cells (FIG. 6b), LNCaP-EGFR cells (FIG. 6c) and LNCaP-erbB2 cells (FIG. 6d). Cells were treated with JDF-12, MTZ+JDF04R (1:1) or MTZ+Iressa (1:1) over a period of 6 days (continuous exposure). Growth inhibition was measured using the SRB assay. Each point represents at least two independent experiments run in triplicate.

FIG. 7a shows the Annexin V binding following drug treatment in the DU145 cell line: (a) annexin V staining; (e) PI staining; (b-d) cells treated with JDF12 at varying concentrations; (f-h) cells treated with JDF04R; (i-k) cells treated with MTZ at varying concentrations. FIG. 7b shows the assessment of late apoptosis in DU145 cells induced by JDF-12, JDF04R, MTZ or Iressa. FIG. 7c shows the assessment of total apoptosis in DU145 cells induced by JDF-12, JDF04R, MTZ or Iressa. The cells were exposed to drug treatment over a period of 48 h. Each point represents two independent experiments run in duplicate.

FIG. 8 shows the in vivo antitumor activity of JDF12 (8a) and Iressa (8b) against the DU145 cell line in CD-1 mice; FIG. 8c shows the weight loss in CD-1 mice following treatment with JDF12. The mice were implanted with the DU145 cell line and were randomly divided into 3 groups. The first group was treated with vehicle (control), the second group was treated with JDF12 and the third group was treated with Iressa. 31 days following treatment, a significant difference in tumor volume could be observed between the group that received JDF12 and the group that received the control. Statistical analysis was carried out using Student's t test (p=0.0289 JDF12 vs. vehicle).

### DETAILED DESCRIPTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The term “derivative” as used herein, is understood as being a substance which comprises the same basic carbon skeleton and carbon functionality in its structure as a given compound, but can also bear one or more substituents or rings.

The term “prodrug”, as used herein, is understood as being a compound which, upon administration to a subject, undergoes chemical conversion by metabolic or chemical processes to yield a compound of Formula I or Formula II, or a salt and/or solvate thereof. Solvates of the compounds of Formula I or II are preferably hydrates.

The term “analogue” as used herein, is understood as being a substance which does not comprise the same basic carbon skeleton and carbon functionality in its structure as a “given compound”, but which can mimic the given compound by incorporating one or more appropriate substitutions such as for example substituting carbon for heteroatoms.

The term “salt(s)” as used herein, is understood as being acidic and/or basic salts formed with inorganic and/or organic acids or bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps.

As used herein, the term “pharmaceutically acceptable salts” include the acid addition and the base salts of the combi-molecules of the present disclosure. Non-limiting acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate (i.e. mesylate), ethanesulphonate, benzenesulphonate, p-toluenesulphonate and pamoate salts. Non-limiting base addition salts are formed from bases which form non-toxic salts and examples are the sodium, potassium, aluminum, calcium, magnesium, zinc and diethanolamine salts.

As used herein, the term “alkyl” can be straight-chain or branched. Examples of alkyl residues containing from 1 to 6 carbon atoms are methyl, ethyl, propyl, butyl, pentyl, hexyl, the n-isomers of all these residues, isopropyl, isobutyl, isopentyl, neopentyl, isohexyl, 3-methylpentyl, sec-butyl, tert -butyl, or tert-pentyl.

The present description refers to a number of chemical terms and abbreviations used by those skilled in the art. Nevertheless, definitions of selected terms are provided for clarity and consistency.

EGFR: Epidermal Growth Factor Receptor; TK: Tyrosine Kinase; EGF: Epidermal Growth Factor; DMSO: Dimethyl Sulfoxide; FBS: Fetal Bovine Serum; SRB: Sulforhodamine B; PGT, Poly(L-Glutamic acid-L-Tyrosine, 4:1); PBS: Phosphate-Buffered Saline; HRP: Horseradish Peroxidase; ELISA: Enzyme-Linked Immunosorbent Assay; PDGF: Platelet-Derived Growth Factor; MAPK: Mitogen-Activated Protein Kinase; Erk1, 2: Extracellular Signal-Regulated Kinase 2; TGFa: Transforming Growth Factor alpha; AGT: O⁶-Alkylguanine Transferase; PI: Propidium Iodide.

The present disclosure relates to molecules referred to as “combi-molecules” that exhibit a dual mode of anti-tumor action; blocking epidermal growth factor receptor (EGFR) mediated signal transduction and damaging DNA by alkylation. In an embodiment, the, present disclosure relates to molecules that exhibit inherent receptor affinity and which upon degradation under physiological conditions produce a metabolite exhibiting receptor affinity for the same receptor as the parent molecule while concomitantly producing a DNA damaging species. In a further embodiment, the present specification relates to molecules capable of antiproliferative activity in tumor cells. In yet a further embodiment, the present specification relates to combi-molecule JDF12 which is an inhibitor of EGFR and which upon degradation under physiological conditions produces a further inhibitor of EGFR (JDF04R) as well as a DNA alkylating moiety.

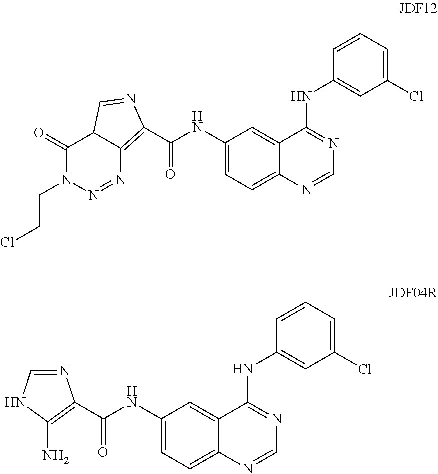

Since the DNA damaging moiety generated by JDF12 upon degradation is identical to that generated by MTZ, it was expected that it would induce single strand breaks and more importantly DNA cross-links, a type of lesion believed to be associated with the superior hematotoxicity of MTZ when compared to its methylating counterpart temozolomide (TMZ). Interestingly, MTZ induced an approximately 2-fold higher level of DNA damage than JDF12. This may be due to a slower rate of degradation of JDF12 inside the cells. On the other hand, and in contrast to MTZ, JDF12 induced a dose dependent inhibition of EGFR phosphorylation inside the cells in addition to producing stronger levels of apoptosis than Iressa and JDF04R. Despite its stronger DNA damaging potential, MTZ induced lower levels of apoptosis than JDF12 indicating that the EGFR inhibitory function of JDF12 plays a key role. Indeed, JDF12 induced significantly higher levels of apoptosis than any of the single-function molecules (e.g. MTZ, Iressa).

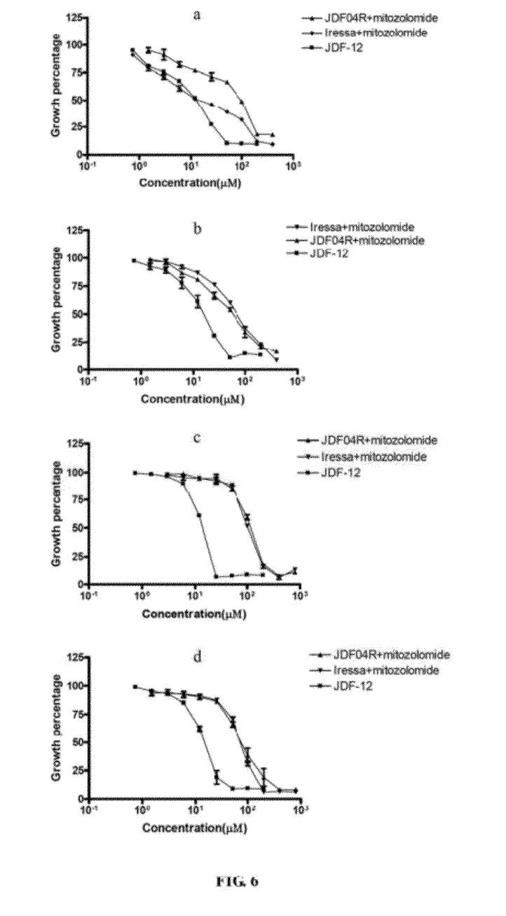

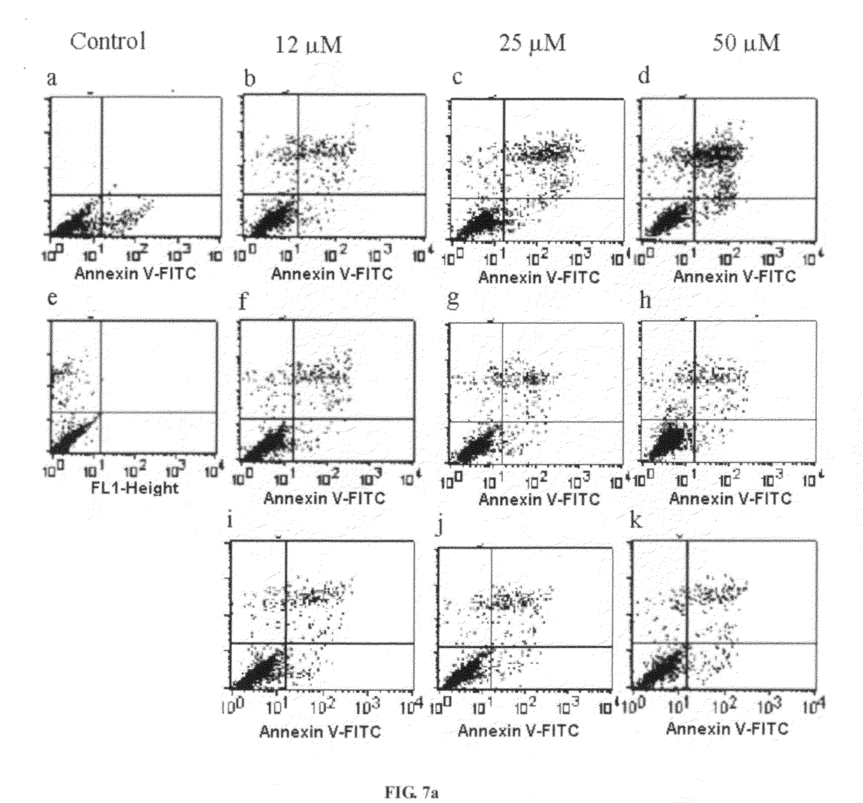

Having demonstrated that JDF12 could damage DNA, block EGFR and induce significantly higher levels of apoptosis, its cytotoxic advantage over equieffective and/or equimolar drug combinations of MTZ and an EGFR inhibitor was determined in the DU145, LNCaP WT, LNCaPEGFR and LNCaPerbB2 cell lines. The superior potency of the combined EGFR TK inhibitory and DNA damaging properties of JDF12 over combinations of MTZ+Iressa or MTZ+JDF04R is illustrated in FIGS. 5 and 6. In the CD-1 male nude mouse prostate model, JDF12 showed minor toxicity at 100 mg/kg, a dose at which MTZ was determined to be toxic. Furthermore, the antitumor activity of JDF12 was determined to be superior to that of an equidose of Iressa. JDF12 thus presents a better pharmacological profile than either MTZ or Iressa.

In an embodiment, the present disclosure relates to pharmaceutical compositions comprising a pharmaceutically effective amount of one or more combi molecules as defined herein or pharmaceutically acceptable salts thereof, in association with one or more pharmaceutically acceptable carriers, excipients and/or diluents. The term “pharmaceutically effective amount” is understood as being an amount of combi molecule required upon administration to a mammal in order to induce EGFR inhibition and/or DNA damage (e.g. alkylation). Therapeutic methods comprise the step of treating patients in a pharmaceutically acceptable manner with one or more combi molecules or compositions comprising one or more combi molecules as disclosed herein.

The therapeutic agents of the present disclosure (i.e. combi molecules) may be administered alone or in combination with pharmaceutically acceptable carriers. The proportion of each carrier is determined by the solubility and chemical nature of the agent(s), the route of administration, and standard pharmaceutical practice. In order to ensure consistency of administration, in an embodiment of the present disclosure, the pharmaceutical composition is in the form of a unit dose.

The combi molecules of the present disclosure may be injected parenterally; this being intramuscularly, intravenously, or subcutaneously. For parenteral administration, the combi molecules may be used in the form of sterile solutions containing solutes, for example sufficient saline or glucose to make the solution isotonic.

For parenteral administration, fluid unit dosage forms may be prepared by utilizing one or more combi molecules and a sterile vehicle, and, depending on the concentration employed, the combi molecule(s) may be either suspended or dissolved in the vehicle. Once in solution, the combi molecule(s) may be injected and filter sterilized before filling a suitable vial or ampoule followed by subsequently sealing the carrier or storage package. Adjuvants, such as a local anesthetic, a preservative or a buffering agent, may be dissolved in the vehicle prior to use. Stability of the pharmaceutical composition may be enhanced by freezing the composition after filling the vial and removing the water under vacuum, (e.g., freeze drying). Parenteral suspensions may be prepared in substantially the same manner, except that the combi molecule(s) should be suspended in the vehicle rather than being dissolved and, further, sterilization is not achievable by filtration. The combi molecule(s) may be sterilized, however, by exposing it to ethylene oxide before suspending it in the sterile vehicle. A surfactant or wetting solution may be advantageously included in the composition to facilitate uniform distribution of the combi molecule(s).

The pharmaceutical compositions of the present disclosure comprise a pharmaceutically effective amount of one or more combi molecules as described herein and one or more pharmaceutically acceptable carriers, excipients and/or diluents.

In an embodiment of the present disclosure, the pharmaceutical compositions contain from about 0.1% to about 99% by weight of a combi molecule as disclosed herein. In a further embodiment of the present disclosure, the pharmaceutical compositions contain from about 10% to about 60% by weight of a combi molecule as disclosed herein, depending on which method of administration is employed. Physicians will determine the most-suitable dosage of the present therapeutic agents (i.e. combi molecules). Dosages may vary with the mode of administration and the particular combi molecule chosen. In addition, the dosage may vary with the particular patient under treatment. The dosage of the combi molecule used in the treatment may vary, depending on the condition, the weight of the patient, the relative efficacy of the compound and the judgment of the treating physician.

### General Synthesis of Combi-Molecules

Combi-molecules JDF11, JDF12, JDF17 and JDF18 were prepared as shown hereinbelow in Scheme 1. MTZ conjugates JDF12 and JDF18 were obtained by treatment of corresponding amines with the acyl chloride referred to as MTZ-OCl in the presence of DIPEA. The glycine or sarcosinamide spacers were introduced by peptide coupling with the corresponding N-protected amino acid, Boc-Gly-OH or Boc-Sar-OH. The Boc protecting groups were removed under acidic conditions to provide the corresponding free amino functions were subsequently coupled with MTZ-OCl in the presence of DIPEA to yield JDF11 and JDF17.

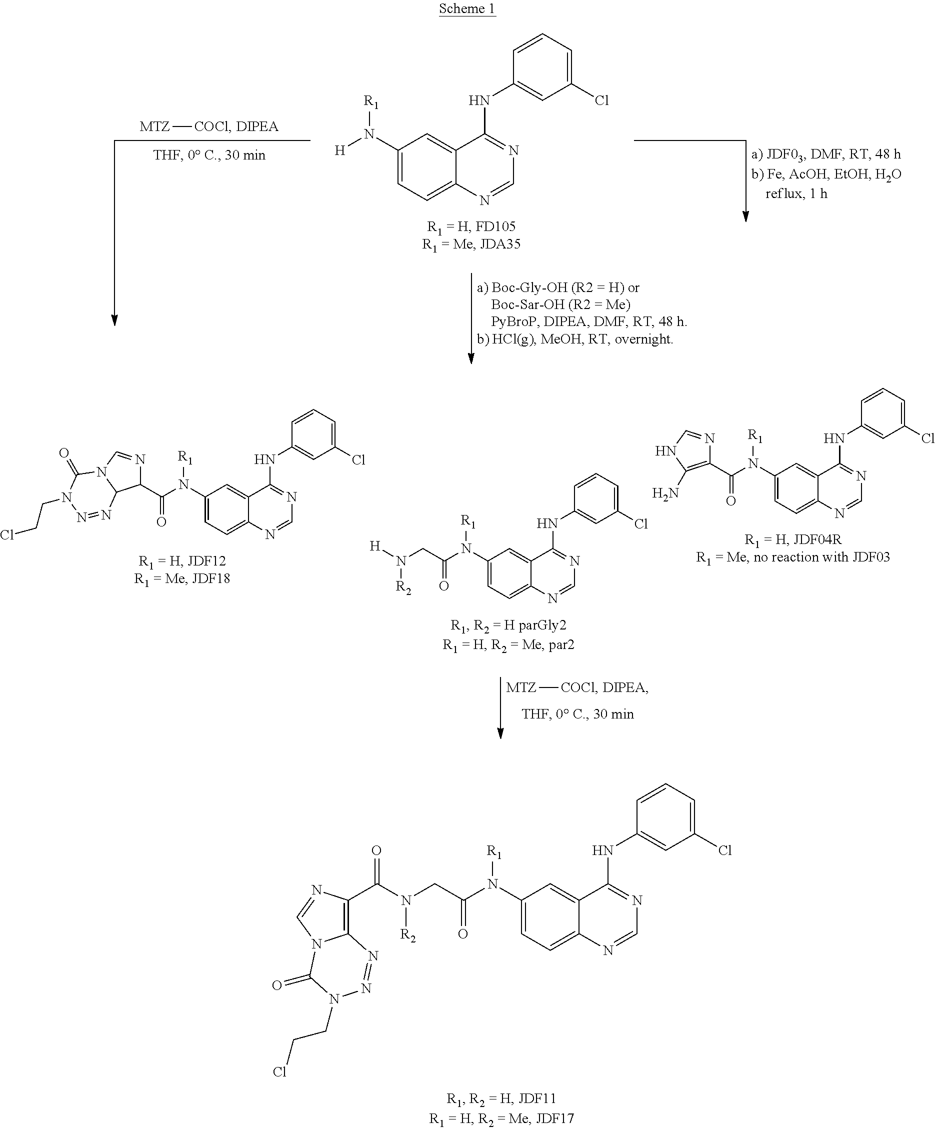

The key synthons, MTZ, its acyl chloride derivative MTZ-OCl and the structure labeled as JDF03 were prepared according to known literature procedures (Scheme 2) [11,23]. Briefly, 5-aminoimidazole-4-carboxamide was treated with NaNO₂ and the resulting product reacted with 2-chloroethylisocyanate in ethyl acetate to provide MTZ. The amide function was hydrolyzed by treatment with NaNO₂ in sulfuric acid and the resulting carboxylic acid function treated with SOCl₂ to provide MTZ-OCl. JDF03 was prepared from nitroimidazole carboxylic acid by treatment with SOCl2. The nitroimidazole carboxylic acid was readily obtained by nitration of imidazole carboxylic acid (Scheme 2) as previously described [11,23].

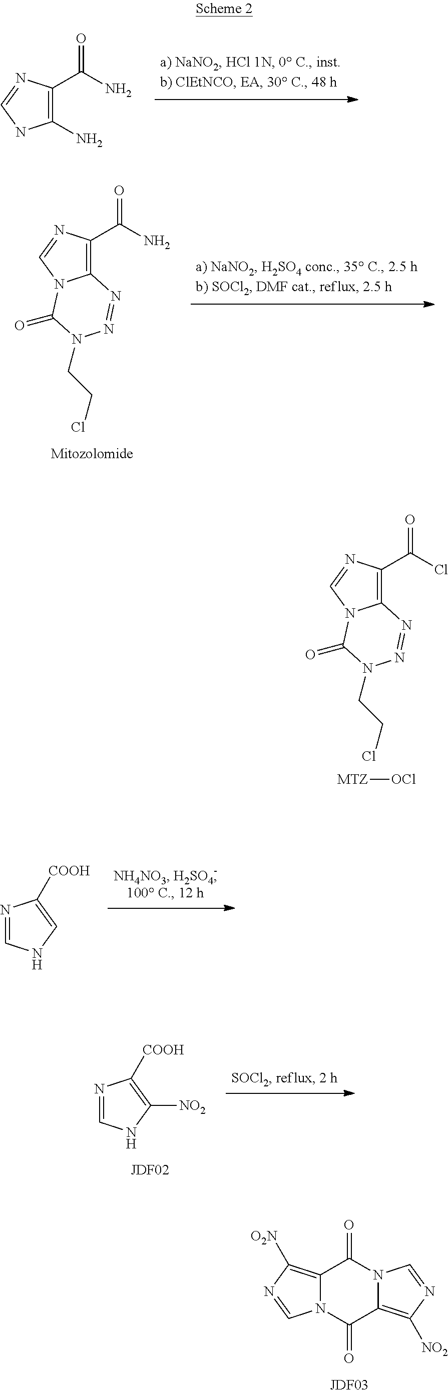

### Materials and Methods

Drug Treatment: JDF12 and JDF04R were synthesized as described in the present disclosure. MTZ was obtained from Developmental Therapeutic Program NCI/NIH (sample reference: 35345l-T/6/0, Lot #AJ 111.1). Iressa was purchased from AstraZeneca (Pharmaceuticals, Mississauga, Ontario, Canada). In all assays, the drug was dissolved in DMSO and subsequently diluted in sterile RPMI 1640 containing 10% fetal bovine serum (Wisent, St. Bruno, Quebec, Canada) immediately before treatment of cell cultures [concentration of DMSO never exceeded 0.2% (v/v)].

Cell Culture: The human prostate cancer cell line DU145 was obtained from the American Type Culture Collection (Manassas, Va.)). The androgen-sensitive prostate cancer cell lines LNCaP-FGC, LNCaP-EGFR and LNCaP-erbB2 (stably transfected with EGFR erbB2 gene respectively) were generous gifts from Dr. Moulay Aloui-Jamali (Jewish General Hospital, Montreal, Quebec, Canada). All the cell lines were maintained in RPMI 1640 supplemented with 10% fetal bovine serum and antibiotics [16]. Cells were maintained in a monolayer at 37° C. in a humidified environment composed of 5% CO₂ and 95% air. The cultures were maintained in logarithmic growth by harvesting with a trypsin-EDTA solution containing 0.5 mg/mL trypsin and 0.2 mg/mL EDTA and replating before confluence. In all assays, the cells were plated over a period of time ranging from 24-48 h prior to drug administration.

Degradation: JDF12 was dissolved in a minimum volume of DMSO, diluted with RPMI-1640 supplemented with 10% FBS and incubated over a period of 22 h at 37° C. Following evaporation to dryness, the samples were reconstituted in acetonitrile and analyzed using liquid chromatography-mass spectrometry. Purified standards [analytes, including metabolites and internal standard] were fragmented, and the appropriate product ions selected for multiple reaction monitoring. Linear standard curves (R>0.99) were obtained between 1.00 and 100 ng for each standard. Samples were injected on a Zorbax C18 (150×4.6 mm, 5 μm) column, and the effluent was introduced into a Sciex API III mass spectrometer. All samples were quantitated relative to the standard curves.

EGFR Kinase Enzyme Assay: The EGFR kinase assay was performed following a known procedure [17]. Briefly, Nunc MaxiSorp (Nalge Nunc International, Rochester, N.Y., USA) 96-well plates were incubated overnight at 37° C. with 100 μL/well of 0.25 ng/mL poly(L-glutamic acid-L-tyrosine, 4:1) PGT in PBS. Excess PGT was removed and the plates were washed three times with wash buffer (0.1% Tween-20 in PBS). The kinase reaction was performed using commercially available isolated EGFR (4.5 ng/well). The compound was added and phosphorylation was initiated by the addition of 20 μL/well of ATP (50 μM). After 8 min at room temperature with constant shaking, the reaction was terminated by aspiration of the reaction mixture and rinsing of the plates (four times) with wash buffer. Phosphorylated PGT was detected following a 25 min incubation with 50 μL/well of horseradish peroxidase-conjugated PY20 anti-phosphotyrosine antibody (Santa Cruz Biotechnology, Santa Cruz, Calif., USA) diluted to 0.2 mg/mL in blocking buffer (3% bovine serum albumin and 0.05% Tween-20 in PBS). The antibody was removed by aspiration and the plates washed four times with wash buffer. The signals were developed by the addition of 50 μL/well of 3,3′,5,5′-tetramethylbenzidine peroxidase substrate (Kirkegaard and Perry Laboratories, Gaithersberg, Md., USA). Following blue color development, H₂SO₄ (50 μL; 0.09 M) was added per well to stop the reaction. The plates were read at 450 nm using a Bio-Rad ELISA reader (model 2550; Bio-Rad, Hercules, Calif., USA).

Growth Inhibition Assay: To study the effect of the compounds (e.g. JDF12 and JDF04R) on serum-stimulated proliferation, cells were grown to 70% confluence in 96-well plates. Cell monolayers were subsequently continuously exposed to different concentrations of each drug over a period of 6 days. For the combination of MTZ and JDF04R or MTZ and Iressa, the drugs were mixed at 1:1 ratio for equal molecule combination or at their IC₅₀ ratio for equal effect combination, serially diluted and added to the monolayers over a period of 6 days. All cell growth-inhibitory activities were evaluated using the sulforhodamine B (SRB) assay [18]. Briefly, following drug treatment, cells were fixed using 50 μL of cold trichloroacetic acid (50%) over a period of 60 minutes at 4° C., washed four times with tap water, and stained over a period of 30 minutes at room temperature using sulforhodamine B (0.4%) dissolved in acetic acid (0.5%). The plates were rinsed five times with 1% acetic acid and allowed to air-dry. The resulting colored residue was dissolved in 200 μL of Iris base (10 mM) and the optical density read for each well at 492 nm using a Bio-Rad microplate reader (model 2550). GraphPad Prism software (version 4.03; San Diego, Calif.) was used for statistical analysis. P<0.05 was considered to indicate statistical significance. The concentration that inhibits 50% (IC₅₀) was analyzed by nonlinear regression analysis (sigmoidal dose-response curve fit). For equal effect combination analysis, IC₅₀ values were determined using the median effect equation as described by Perez et al. [19]. The combination index (CI) was calculated according to the following formula:

$$C I = \frac{{IC}_{50} ( Drug 1 in Combination )}{{IC}_{50} ( Drug 1 Alone )} + \frac{{IC}_{50} ( Drug 2 in Combination )}{{IC}_{50} ( Drug 2 Alone )}$$

CI values>1, =1, and <1 indicate antagonism, additivity and synergism respectively. Each point represents the average of at least two independent experiments run in triplicate.

EGFR Phosphorylation Assay: DU145 cells (1×10⁶) were preincubated in a six-well plate using 10% serum at 37° C. over a period of 24 h followed by a starving period of 24 h. The cells where then exposed to a dose range of each drug over a period of 2 h and subsequently treated with 50 ng/ml of EGF for 20 min at 37° C. The cells were washed with PBS and resuspended in cold lysis buffer [50 mmol/L Tris-HCl (pH 7.5), 150 mmol/L NaCl, 1% NP40, 1 mmol/L EDTA, 5 mmol/L NaF, 1 mmol/L Na₃VO₄, protease inhibitor tablet (Roche Biochemicals, Laval, Quebec, Canada)]. The lysates were kept on ice for 30 min and collected by centrifugation at 10,000 rpm for 20 min at 4° C. The concentrations of protein were determined using the Bio-Rad protein assay kit (Bio-Rad Laboratories, Hercules, Calif.). Equal amounts of protein (50 mg) were subjected to a 10% SDS-polyacrylamide gel electrophoresis (SDS-PAGE) and transferred to a polyvinylidene difluoride membrane (Millipore, Bedford, Mass.). Nonspecific binding on the membrane was minimized with a blocking buffer containing bovine serum albumin (5%) in PBS-Tween 20. The membranes were subsequently incubated with primary anti-phosphotyrosine antibody (Upstate Biotechnology, Lake Placid, N.Y.) for the detection of phosphotyrosine. The membranes were stripped and reprobed with anti-EGFR (Neomarkers, Fremont, Calif.) for determination of corresponding receptor levels. Blots were incubated with horseradish peroxidase-goat anti-mouse antibody (1:1,000 dilution; Cell Signaling Research, Beverly, Mass.), and the bands visualized with an enhanced chemiluminescence system (Amersham Pharmacia Biotech,. Little Chalfont, UK) [20]. Band intensities were measured using the SynGene GeneTools software package.

Alkaline Comet Assay for Quantization of DNA Damage: A modified alkaline comet assay technique was used for quantization of DNA damage induced in DU145 cells [21]. The cells were exposed to a dose range of the drugs (JDF12, JDF04R, or MTZ) over a period of 2 h and harvested with trypsin-EDTA. The cells were subsequently collected by centrifugation and resuspended in PBS. The resulting cell suspensions were diluted to 3×10⁵ cell/mL and mixed with agarose (0.75%) in PBS at 37° C. in a 1:9 dilution. The gels were cast on Gelband strips (Mandel Scientific, Guelph, Ontario, Canada) using gel casting chambers and then immediately placed into a lysis buffer (2.5 M NaCl, 0.1 M tetrasodium EDTA, 10 mM Trisbase, 1% (v/v) N-lauryl sarcosine, 10% (v/v) DMSO, and 1% (v/v) Triton X-100). After being kept overnight at 4° C., the gels were gently rinsed with distilled water and immersed in a second lysis buffer (2.5 M NaCl, 0.1 M tetrasodium EDTA, 10 mM Tris base) containing 1 mg/mL proteinase K for 60 min at 37° C. The cells were subsequently rinsed with distilled water, incubated in alkaline electrophoresis buffer for 30 min at 37° C. and electrophoresed at 400 mA for 20 min. The gels were then rinsed with distilled water and placed into 1 M ammonium acetate for 30 min. The gels were subsequently soaked in 100% ethanol for 2 h, dried overnight and stained with SYBR Gold (1:10000 dilution of stock supplied by Molecular Probes, Eugene, Oreg.) for 20 min. Comets were visualized at 330× magnification and DNA damage was assessed using the tail moment parameter (i.e. the distance between the barycenter of the head and the tail of the comet multiplied by the percentage DNA in the tail region). A minimum of 50 cell comets were analyzed for each sample, using Comet Assay χ v4.2 image analysis software, and the values presented as average tail moments for the entire cell population.

Annexin V binding Analysis: Cells were pre-incubated in 6-well plates until confluence and then exposed to a dose range of each drug over a period 48 h. The cells were subsequently harvested and incubated with annexin V-FITC and propidium iodide (PI) using the apoptosis Detection Kit (BD Bioscience Pharmingen, USA) and the supplier's protocol. Annexin V-FITC and PI binding were analyzed using a Becton-Dickinson FACScan. Data were collected using logarithmic amplification of both the FL1 (FITC) and FL2 (PI) channels. Quadrant analysis of coordinate dot plots was performed with CellQuest software.

Mouse Xenograft Studies (DU1451: CD1 male mice (Charles River) were maintained according to the McGill guidelines for the use of laboratory animals. Dose finding was done with two mice per group and the maximum tolerated dose was defined as the dose that does not induce >15% weight loss over a period of at least 14 days. For xenograft studies, mice were treated with 1×10⁶ cells suspended in 0.2 mL PBS injected s.c. into the flank of each mouse. Treatments began when tumors became palpable (n=6 mice/group). Each drug (100 mg/kg) was given by intraperitoneal (i.p.) injection in an aqueous solution of Cremophore (25%)/ethanol (25%) (0.2 ml) every other day over a period of 1 month. The tumor burden was measured with a caliper before each injection and the tumor volume calculated using the formula:

$$T V = {[ \frac{Tumor Width + Tumor Length}{4} ]}^{3} \times 4 / 3 \times π$$

Statistical analysis was carried using two-tailed Student's t-test.

Degradation of JDF12: Under physiological conditions, JDF12 is expected to decompose via ring opening of the tetrazinone moiety to yield the monochloroethyl triazenylimidazole carboxamide that further degrades into aminoimidazole (JDF04R) and chlorethyldiazonium (Scheme 3).

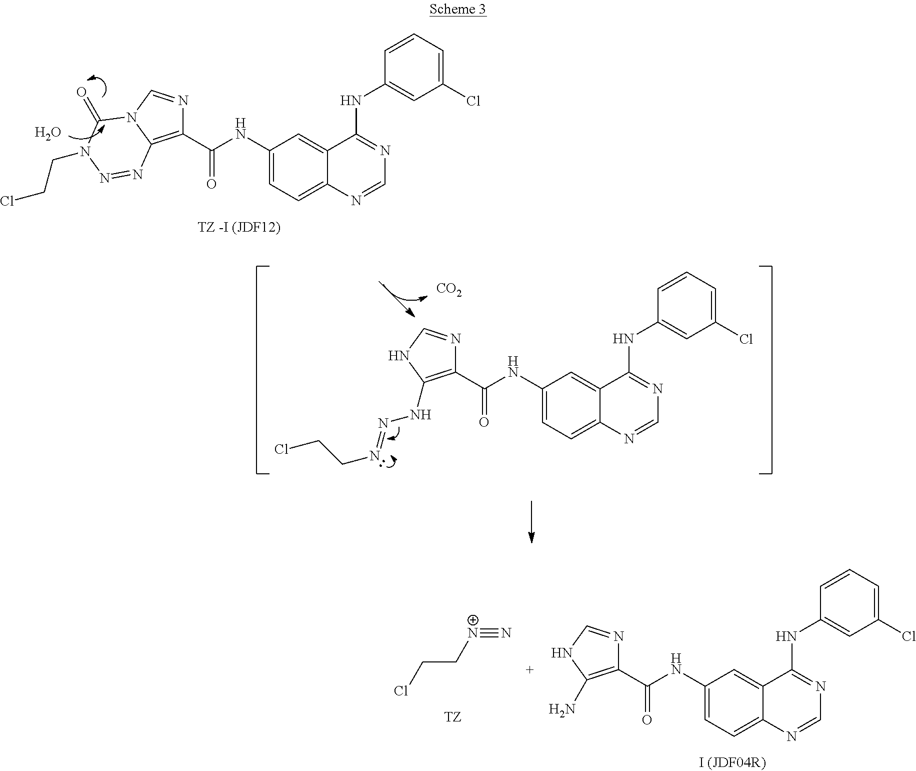

JDF12 was allowed to decompose in cell culture medium at 37° C. The degradation products were subsequently analyzed by LCMSMS. The results showed the presence of JDF04R thus confirming ring opening of the tetrazinone moiety. JDF04R was formed in a calculated overall yield of 71%. Thus, in vitro incubation of JDF12 over periods exceeding 72 h results in JDF04R being the major product present in the medium. Independently synthesized JDF04R was tested in almost all biological evaluations for the purpose of comparison.

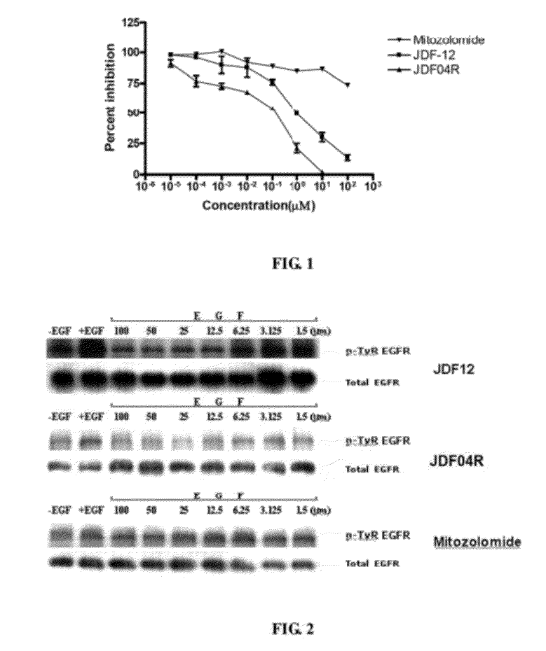

Inhibition of EGFR TK: The EGFR TK inhibitory activity of JDF12 and its metabolite JDF04R was tested in a short 8 minute exposure to an ELISA assay. The assay was based on the inhibition of phosphorylation of poly(L-glutamic acid-L-tyrosine, 4:1) (PGT) by EGFR tyrosine kinase. The results showed IC₅₀ values of 0.62 μM for JDF12 and of 0.23 μM for JDF04R. The results are indicative of JDF12 being a prodrug of the more potent EGFR TK inhibitor JDF04R (2.7 fold). As expected, the EGFR TK-inhibitory activity of MTZ was negligible (FIG. 1).

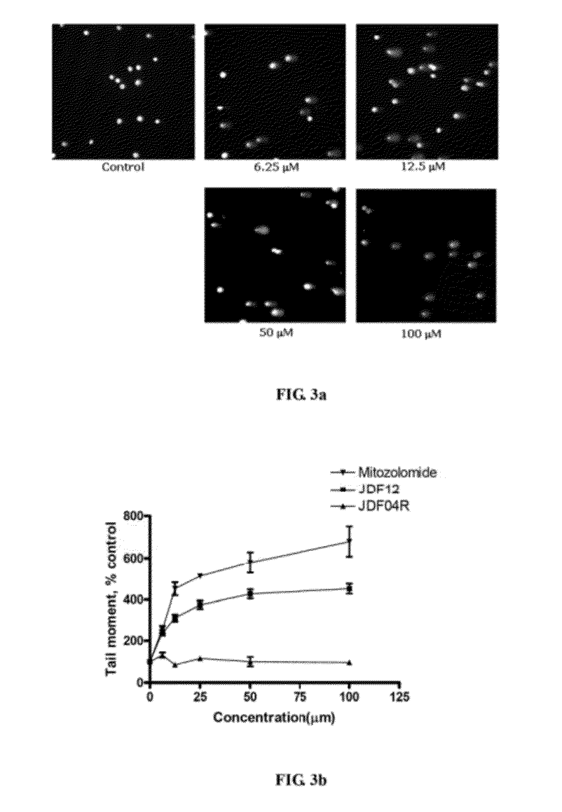

Inhibition of EGFR TK in Whole Cell Assay: DU145 cells were starved for 24 h and stimulated with EGF in the presence of the different drugs over a period of 2 h. Inhibition of autophosphorylation of EGFR was analyzed by Western blotting. The observed order of potency from this whole cell assay paralleled the one previously observed with ELISA. In contrast to MTZ, JDF12 blocked EGFR autophosphorylation in a dose-dependent manner (IC₅₀ 12 μM). However, its potency was approximately 2-fold less than that observed for JDF04R which induced an almost 100% inhibition of EGFR TK at concentrations as low as 6 μM (FIG. 2). These results further corroborate that JDF12 releases an even more potent inhibitor of EGFR (i.e. JDF04R).

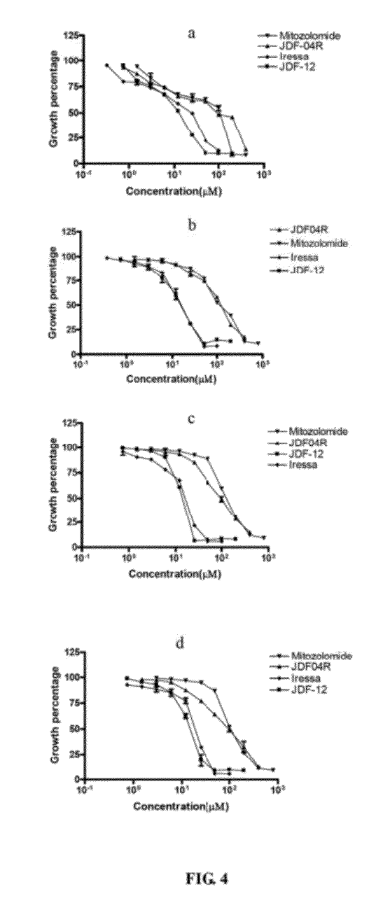

DNA Damaging Properties of JDF12: The EGFR TK inhibitor JDF12 was shown to decompose under physiological conditions to yield the even more potent EGFR TK inhibitor JDF04R while concomitantly generating the DNA damaging agent chlorethyldiazonium (Scheme 1). The reactive nature of the latter species makes its detection very difficult. However, the induction of DNA damage (i.e. DNA alkylation) represents evidence of its formation and fleeting presence. The alkaline comet assay was used to demonstrate that much like MTZ, JDF12 induced dose-dependent DNA damage in DU145 cells following exposure to the drug over a period of 2 hours (FIG. 3a-b). Interestingly, JDF12 induced a 2-fold less DNA damage than MTZ. As expected, the EGFR inhibitor JDF04R did not demonstrate any DNA damaging activity.

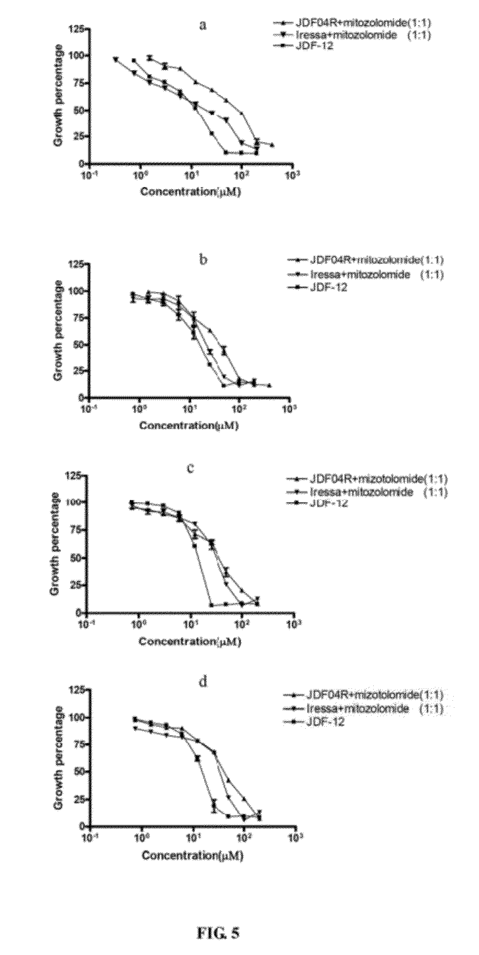

Antiproliferative Activity: In contrast to its metabolite JDF04R, its imidazotetrazinone counterpart MTZ or the EGFR TK inhibitor Iressa (which was shown to exhibit significant antitumor activity), the binary EGFR/DNA targeting of JDF12 translated into a 10-16-fold increased anti-proliferation potency when compared to MTZ and a 2-3-fold increased anti-proliferation potency when compared to Iressa against the DU145, LNCaP, LNCaPEGFR, and LNCaPerb2 cell lines (FIG. 4a-d). The results are further illustrated hereinbelow in Table 1.

TABLE 1
IC₅₀ values for JDF12, JDF04R, MTZ and Iressa (μM)
(x ± SD) against various cell lines.

| Cell Type | JDF-12 | JDF04R | MTZ | Iressa |
|---|---|---|---|---|
| DU145 | 11.28 ± 0.22 | 151.05 ± 17.04 | 178.75 ± 21.14 | 31.45 ± 2.06 |
| LNCaP-WT | 13.19 ± 3.88 | 177.90 ± 13.29 | 150.40 ± 7.92 | 20.03 ± 0.44 |
| LNCaP-EGFR | 11.99 ± 0.57 | 119.40 ± 4.10 | 175.70 ± 6.51 | 20.34 ± 1.39 |
| LNCaP-ErbB2 | 13.26 ± 2.02 | 111.05 ± 18.05 | 142.45 ± 8.93 | 31.83 ± 4.00 |

JDF12 being a molecule not only capable of blocking EGF-stimulated signal transduction on its own but also of generating a DNA alkylating species that may inflict irreversible cytotoxic lesions (i.e. binary EGFR/DNA targeting properties) in addition to releasing a further EGFR TK inhibitory molecule (JDF04R), the potency of JDFI2 was compared to that of an equimolar drug combination comprising MTZ and JDF04R. Moreover, the potency of JDFI2 was compared to an equimolar combination of the well-known EGFR TK inhibitor Iressa and MTZ. The results showed that JDF12 was a 1.7-fold more potent inhibitor than an equimolar combination of MTZ with either Iressa or JDF04R against the DU145, LNCaP, LNCaPEGFR, and LNCaPerb2 cell lines (FIG. 5a-d) suggesting that the single combi-molecule JDF12 is a superior mimic of a two-drug combination. The results are further illustrated hereinbelow in Table 2.

TABLE 2
IC₅₀ values for JDF12, JDF04R/MTZ (equimolar combination)
and Iressa/MTZ (equimolar combination) (μM) (x ±
SD) against various cell lines.

| Cell Type | JDF-12 | MTZ + JDF04R | MTZ + Iressa |
|---|---|---|---|
| DU145 | 11.28 ± 0.22 | 75.50 ± 4.89 | 19.19 ± 4.07 |
| LNCaP-WT | 13.19 ± 3.88 | 33.75 ± 3.95 | 23.20 ± 1.51 |
| LNCaP-EGFR | 11.99 ± 0.57 | 49.57 ± 10.95 | 38.73 ± 0.81 |
| LNCaP-ErbB2 | 13.26 ± 2.02 | 67.80 ± 6.35 | 48.54 ± 1.01 |

To further demonstrate the efficacy of JDF12, its potency was compared to that of an equieffective drug dosage combination comprising MTZ and JDF04R and an equieffective drug dosage combination comprising Iressa and MTZ. The drug dosage combinations were comprised of IC₅₀ (EGFR inhibitor)/IC₅₀ (MTZ). The results showed that the MTZ+JDF04R or MTZ+Iressa combination resulted in synergistic activity in the DU145, LNCaP, LNCaPEGFR, and LNCaPerb2 cell lines with a CI50<1 (FIG. 6a-d). For example, the results showed that the combination index (CI) at the 50% effect (CI50) for the JDF04R+MTZ combination is 0.68, indicating a subadditive interaction. However, under identical conditions, the antiproliferative activity of JDF12 was more than 1.3-fold more potent than that of the equieffective 2-drug combination. The results are further illustrated hereinbelow in Table 3.

TABLE 3
IC₅₀ values for JDF12, JDF04R/MTZ (equieffective
combination) and Iressa/MTZ (equieffective combination)
(μM) (x ± SD) against various cell lines.

| Cell Type | JDF-12 | MTZ + JDF04R | MTZ + Iressa |
|---|---|---|---|
| DU145 | 11.28 ± 0.22 | 111.30 ± 3.54 | 18.97 ± 0.38 |
| LNCaP-WT | 13.19 ± 3.88 | 46.32 ± 8.45 | 86.84 ± 6.87 |
| LNCaP-EGFR | 11.99 ± 0.57 | 142.25 ± 1.34 | 127.60 ± 0.42 |
| LNCaP-ErbB2 | 13.26 ± 2.02 | 109.43 ± 10.94 | 82.86 ± 8.76 |

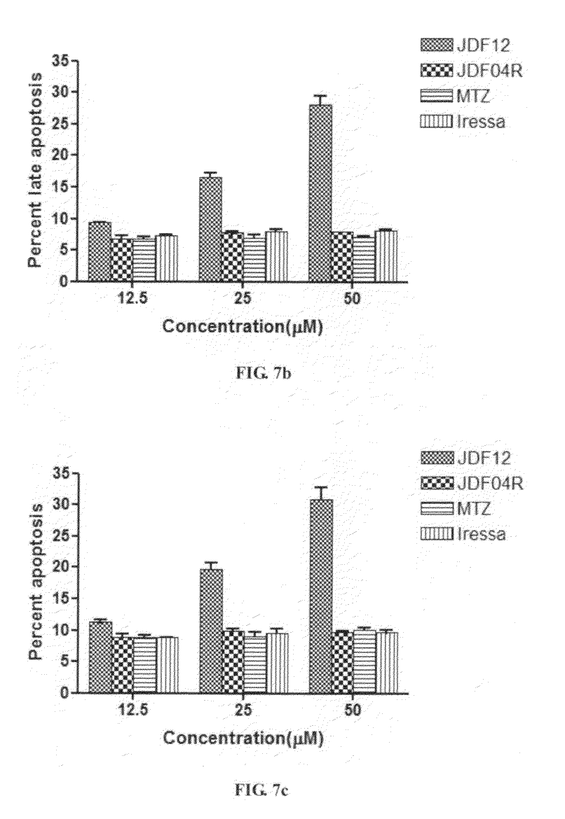

Induction of Apoptosis: Annexin V-FITC (FL1-H) and PI (FL2-H) staining (detected by flow cytometry) was used to distinguish viable (PI-/FITC−), early apoptotic (PI-/FITC+), late apoptotic (PI+/FITC+) and necrotic (PI+/FITC−) cells. At a concentration of 25 μM and following an exposure time of 48 h, JDF12 induced a significant increase in late apoptosis in DU145 cells. JDF04R, Iressa and MTZ however, induced only barely detectable levels of apoptosis (<10%). The results are indicative that the combined EGFR TK inhibitory and DNA damaging properties of JDF12 can induce more cell apoptosis than observed with a single-function drug (e.g. Iressa and MTZ) alone (FIG. 7a-c).

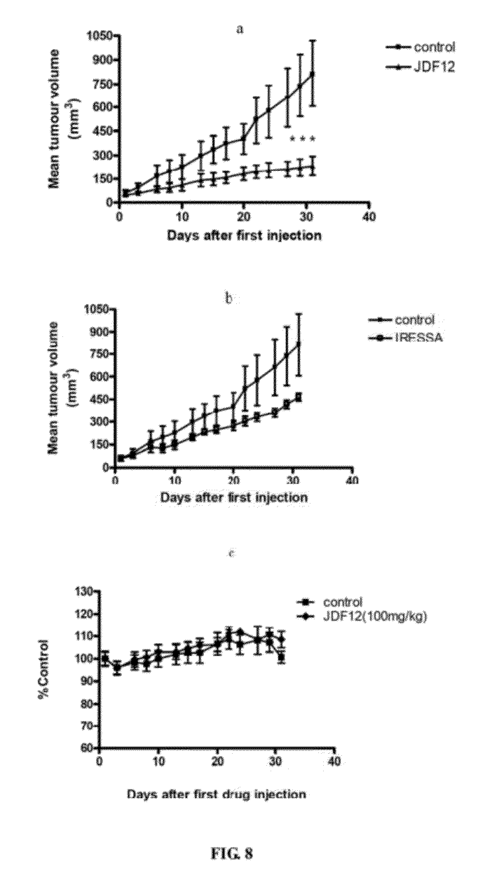

In Vivo Efficacy: A dose finding study was performed by monitoring the weight loss over a 14-day period in groups of two animals (mice). From this study, a 100 mg/kg dose was defined as the maximum tolerated dose for JDF12 (MTZ being toxic at this dose). JDF12 induced significant antitumor activity (P<0.05) 31 days after treatment (FIG. 8a). Iressa did not show significant antitumor activity 31 days after treatment (FIG. 8b).

A comparative toxicity study in CD-1 mice treated with 100 mg/kg of JDF12, Iressa and Mitozolomide was performed (dosing schedule: once every other day). The results are further illustrated hereinbelow in Table 4.

TABLE 4
Comparative toxicity study in CD-1 mice (n =
6) treated with JDF12, Iressa and Mitozolomide.

| | Mitozolomide | Iressa | JDF12 |
|---|---|---|---|
| Day | (Vital Sign) | (Vital Sign) | (Vital Sign) |
| 1 | Normal | Normal | Normal |
| 3 | Normal | Normal | Normal |
| 6 | (BCS = 2) | Normal | Normal |
| | Weight loss > 20% (1 mouse) | | |
| | Death (1 mouse) | | |
| 8 | (BCS = 2) | Normal | Normal |
| | Weight loss > 20% (1 mouse) | | |
| 10 | (BCS = 2) | Normal | Normal |
| | Weight loss > 20% (1 mouse) | | |
| 16 | (BCS = 2) | Normal | Normal |
| | All Mice Thin | | |
| | Death (3 mice) | | |

Normal (i.e. No sign of Toxicity); BCS (Body Condition Score) = 2 indicates that mice are underconditioned (observable signs include segmentation of the vertebral column, palpability of the dorsal pelvic bones).

In contrast to Mitozolomide, JDF12 could be tolerated at a dose as high as 100 mg/kg. Importantly, this level of tolerance parallels that of the clinical EGFR inhibitor Iressa which does not contain a cytotoxic DNA alkylating function. As shown in Table 4, no signs of toxicity were observed for JDF12 and Iressa at the 100 mg/kg dose. By contrast, 6-10 days after initiating treatment, animals treated with Mitozolomide showed signs of dehydration and weight loss with an overall body condition score (BCS) of 2 [22]. No weight loss was observed for JDF12 during the efficacy study (FIG. 8c). The conjugation of the imidazotriazinone backbone of Mitozolomide with an EGFR targeting quinazoline moiety thus presents the advantage of significantly altering its toxicity profile.

Synthesis of JDF12: 6-Amino-4-anilinoquinazoline (FD105; 55 mg, 0.2 mmol) was stirred in dry CH₂Cl₂ (10 mL) under argon with DIPEA (106 μL, 0.6 mmol) and dry pyridine (16 μl, 0.2 mmol). The solution was subsequently cooled to 0° C., and MTZ-COCl was added dropwise in CH₂Cl₂ (5 mL). The solution was stirred over a period of 30 min at 0 ° C. and the resulting precipitate collected by filtration. The precipitate was subsequently washed with ether and dried under vacuum to yield the title compound JDF12 as a pure yellow solid (Yield: 30 mg; 30%). ESI 518 (MNa⁺³⁵Cl³⁵Cl) 520 (MNa⁺³⁵CI³⁷Cl); ¹H NMR (400 MHz, DMSO-d6) δ 10.76 (s, 1H, NH); 9.98 (s, 1H, NH); 9.07 (s, 1H); 8.98 (s, 1H), 8.60 (s, 1H); 8.21 (d, 1H, J=8.4 Hz, H); 8.07 (s, 1H); 7.82 (d, 1H, J=8.8 Hz); 7.40 (t, 1H, j=8.4 Hz); 7.15 (d, 1H, J=8.4 Hz); 4.67 (t, 2H, J=6.0 Hz); 4.04 (t, 2H, J=6.0 Hz).

Synthesis of JDF11: As described hereinabove for JDF12, the title compound JDF11 was obtained as a pure white powder following purification by silica gel chromatography (DCC/Ethanol 93/7) (Yield: 20 mg; 5%). NMR (400 MHz, DMSO-d6) δ 10.46 (s, 1H, NH); 9.93 (s, 1H, NH); 8.94 (s, 1H); 8.76 (t, NH, J=6 Hz), 8.72 (d, 1H, J=1.8 Hz); 8.56 (s, 1H); 8.01 (t, 1H, J=2.1 Hz); 8.88-7.76 (m, 3H); 7.37 (t, 1H, J=8.4 Hz); 7.11 (ddd, 1H, J=0.9 Hz, J=2.1 Hz, J=8.1 Hz); 4.64 (t, 2H, J=6.0 Hz); 4.22 (d, 2H, J=4.03 (t, 2H, J=6.0 Hz).

### REFERENCES

1. Jemal A, Siegel R, Ward E, Murray T, Xu J, Smigal C, Thun M J; Cancer statistics, 2006. CA Cancer J. Clin. 2006, 56:106-130.
2. Hill B, Kyprianou N; Sequencing hormonal ablation and radiotherapy in prostate cancer: A molecular and therapeutic perspective. Oncology Reports 2002, 9:1151-6.
3. Small E J, Fontana J, Tannir N, DiPaola R S, Wilding G, Rubin M, Iacona R B, Kabbinavar F F; A phase II trial of gefitinib in patients with non-metastatic hormone-refractory prostate cancer. BJU Int. 2007, 100:765-9.
4. Kantoff P; Recent progress in management of advanced prostate cancer. Oncology (Williston Park). 2005,19:631-6.
5. Zhang H L, Ye D W, Yao X D, Dai B, Zhang S L, Shen Y J, Zhu Y, Zhang W; Docetaxel plus prednisone versus mitoxantrone plus prednisone for metastatic hormone-refractory prostate cancer in Chinese patients: experience of a single center. Urol Int. 2007,79:307-11.
6. Sirotnak F M, She Y, Lee F, Chen J, Scher H I; Studies with CWR22 xenografts in nude mice suggest that ZD1839 may have a role in the treatment of both androgen-dependent and androgen-independent human prostate cancer. Clin Cancer Res. 2002, 8:3870-3876.
7. Small E J, Fontana J, Tannir N, DiPaola R S, Wilding G, Rubin M, Iacona R B, Kabbinavar F F; A phase II trial of gefitinib in patients with non-metastatic hormone-refractory prostate cancer. BJU Int. 2007, 100:765-9.
8. Canil C M, Moore M J, Winquist E, Baetz T, Pollak M, Chi K N, Berry S, Ernst D S, Douglas L, Brundage M, Fisher B, McKenna A, Seymour L; Randomized phase II study of two doses of gefitinib in hormone-refractory prostate cancer: a trial of the National Cancer Institute of Canada-Clinical Trials Group. J. Clin. Oncol. 2005, 23: 455-60.
9. Pu Y S, Hsieh M W, Wang C W, Liu G Y, Huang C Y, Lin C C, Guan J Y, Lin S R, Hour T C; Epidermal growth factor receptor inhibitor (PD168393) potentiates cytotoxic effects of paclitaxel against androgen-independent prostate cancer cells. Biochem Pharmacol. 2006, 71:751-60.
10. Hickman J A, Stevens M F, Gibson N W, Langdon S P, Fizarnes C, Lavelle F, Atassi G, Lunt E, Tilson R M; Experimental antitumor activity against murine tumor model systems of 8-carbamoyl-3-(2-chloroethyl)imidazo[5,1-d]-1,2,3,5-tetrazin-4(3H)-one (mitozolomide), a novel broad-spectrum agent. Cancer Res. 1985, 45:3008-13.
11. Arrowsmith J, Jennings S A, Clark A S, Stevens M F; Antitumor imidazotetrazines. Conjugation of the antitumor agents mitozolomide and temozolomide to peptides and lexitropsins bearing DNA major and minor groove-binding structural motifs. J Med Chem. 2002, 45:5458-70.
12. Fairbairn L J, Chinnasamy N, Lashford L S, Chinnasamy D, Rafferty J A; Enhancing hemopoietic drug resistance: a rationale for reconsidering the clinical use of mitozolomide. Cancer Gene Ther. 2000, 7:233-9.
13. Pegg A E, Swenn K, Chae M Y, Dolan M E, Moschel R C; Increased killing of prostate, breast, colon, and lung tumor cells by the combination of inactivators of O6-alkylguanine-DNA alkyltransferase and N,N-bis(2-chloroethyl)-N-nitrosourea. Biochem Pharmacol. 1995, 50:1141-8.
14. U.S. Pat. No. 7,572,798.
15. U.S. Patent Application Publication 2006/0003970.
16. Domarkas J, Dudouit F, Williams C; The combi-targeting concept: synthesis of stable nitrosoureas designed to inhibit the epidermal growth factor receptor (EGFR). J. Med. Chem. 2006, 49:3544-52.
17. Rachid Z, Brahimi F, Katsoulas A, Teoh N, Jean-Claude B J; The combi-targeting concept: chemical dissection of the dual targeting properties of a series of “combi-triazenes”. J. Med. Chem. 2003, 46:4313-21.
18. Fricker S P, Buckley R G; Comparison of two colorimetric assays as cytotoxicity endpoints for an in vitro screen for antitumor agents. Anticancer Res. 1996, 16:3755-60.
19. Perez E A, Hack F M, Webber L M, Chou T C; Schedule-dependent synergism of edatrexate and cisplatin in combination in the A549 lung-cancer cell line as assessed by median-effect analysis. Cancer Chemother. Pharmacol. 1993, 33:245-50.
20. Bussolati G, Montemurro F, Righi L, Donadio M, Aglietta M, Sapino A; A modified Trastuzumab antibody for the immunohistochemical detection of HER-2 overexpression in breast cancer. Br. J. Cancer. 2005, 92:1261-7.
21. Qiu Q, Domarkas J, Banerjee R, Katsoulas A, McNamee J P, Jean-Claude B J; Type II combi-molecules: design and binary targeting properties of the novel triazolinium-containing molecules JDD36 and JDE05. Anticancer Drugs. 2007, 18:171-7.
22. Ullman-Culleré M H and Foltz C J; Body condition scoring: a rapid and accurate method for assessing health status in Mice Lab. Animal Sci, 1999, 43, 319-323.
23. Clark A. S. Antitumor imidazotetetazines. 32. Synthesis of novel imidazotetrazinones and related bicyclic heterocycles to probe the mode of action of the antitumour drug temozolomide. J. Med. Chem., 1995, 38, 1493-1504.

What is claimed is:

1. A combi-molecule of Formula I or a pharmaceutically acceptable salt or prodrug thereof: 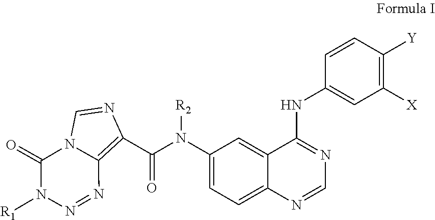
wherein:
a) X is selected from the group consisting of H, F, Cl, Br, I, Me and C≡CH;
b) Y is selected from the group consisting of H, F, Br and Cl;
c) R₁ is selected from the group consisting of H, alkyl; ClCH₂CH₂—, HOCH₂CH₂—, MsOCH₂CH₂—, TsOCH₂CH₂—;
d) R₂ is selected from the group consisting of H, alkyl, ClCH₂CH₂—, HOCH₂CH₂—, MsOCH₂CH₂—, TsOCH₂CH₂—, and 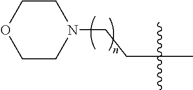
e) n is an integer ranging from 1 to 6.

2. The combi-molecule of claim 1, wherein X is Cl; Y is H; R₁ is ClCH₂CH₂—, and R₂ is H.

3. A combi-molecule of Formula II or a pharmaceutically acceptable salt or prodrug thereof: 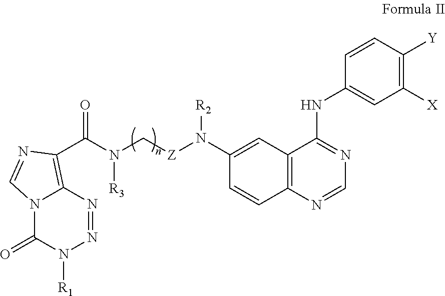
wherein:
a) X is selected from the group consisting of H, F, Cl, Br, I Me and C≡CH;
b) Y is selected from the group consisting of H, F, Br and Cl;
c) Z is selected from the group consisting of —CO— and —CH₂—
d) R₁ is selected from the group consisting of H, alkyl; ClCH₂CH₂—, HOCH₂CH₂—, MsOCH₂CH₂—, TsOCH₂CH₂—;
e) R₂ and R₃ are independently selected from the group consisting of H, alkyl, ClCH₂CH₂—, HOCH₂CH₂—, MsOCH₂CH₂—, TsOCH₂CH₂—, and 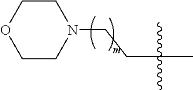
f) n and m are integers ranging from 1 to 6.

4. The combi-molecule of claim 3 wherein X is Cl; Y is H; R₁ is ClCH₂CH₂—, R₂ and R₃ are H; and n is 1.

5. The combi-molecule of claim 3 wherein X is Cl; Y is H; R₁ is ClCH₂CH₂—, R₂ is H; R₃ is Me; and n is 1.

6. A method of treating EGFR expressing tumors comprising administering a therapeutically effective amount of a combi-molecule as defined in claim 1 or 3.

7. A pharmaceutical composition comprising a combi-molecule as defined in claims 1 or 3 and a pharmaceutically acceptable carrier.
